(19) United States
(12) Patent Application Publication
(10) Pub. No.: US 2007/0112061 A1
(43) Pub. Date: May 17, 2007

(54) Heat- or singlet oxygen-generating agents and cancer treatment compositions comprising organic peroxide or chemiluminescent compound

(76) Inventors: Masaru Kimura, Okayama (JP); Hiromi Iwagaki, Okayama (JP); Mitsuru Tsunenaga, Okayama (JP); Shinsuke Inoue, Hiroshima (JP)

Correspondence Address: FRISHAUF, HOLTZ, GOODMAN & CHICK, PC, 220 Fifth Avenue, 16TH Floor, NEW YORK, NY, 10001-7708, US

(21) Appl. No.: 10/593,757

(22) PCT Filed: Mar. 30, 2005
(86) PCT No.: PCT/JP05/06017

§ 371 Date: Sep. 22, 2006

(30) Foreign Application Priority Data
Mar. 31, 2004 (JP) 2004-107084

(51) Int. Cl. A61K 31/337; C07D 305/02; C12Q 1/00

(52) U.S. Cl. 514/449; 549/510; 435/4

(57) ABSTRACT

The present invention provides a heat-generating agent or singlet oxygen-generating agent effective as a new cancer treatment agent which, unlike an anticancer agent based on an alkylating agent such as MMC, uses the action of heat and/or singlet oxygen to kill cancer cells and reduce the burden on patients. A heat- and/or singlet oxygen-generating agent or a cancer treatment agent, comprising an organic peroxide such as the peroxide of an imidazole derivative or a chemiluminescent compound such as a dioxetane compound.

### TECHNICAL FIELD

The present invention relates to a heat- or singlet oxygen-generating agent comprising an organic peroxide or a chemiluminescent compound and to a pharmaceutical composition which, entirely unlike conventional ones, uses heat or singlet oxygen to exhibit anticancer effect.

### BACKGROUND ART

In general, conventional cancer treatments include a method using anticancer drug that is an alkylating agent and a method for generating singlet oxygen using light. However, these methods have such drawbacks that cancer cells easily acquire resistance to the drug and that there are serious side effects. Besides, thermotherapy which has been used conventionally merely warms a patient at a hot spring and is not expected to act directly on cancer cells.

### DISCLOSURE OF THE INVENTION

### Problems to be Solved by the Invention

Therefore, an object of the present invention is to provide a cancer treatment drug which, compared to conventional anticancer drugs, hardly develops above-mentioned side effects and is hardly tolerated, and is capable of reducing such burden on a patient.

### Means for Solving the Problems

The present invention is (1) a heat- and/or singlet oxygen-generating agent comprising an organic peroxide or a chemiluminescent compound.

The present invention is (2) the generating agent according to above-mentioned (1), wherein the generating agent is used for anticancer or inducing sudden death of cells.

The present invention is (3) the generating agent according to above-mentioned (1) or (2), wherein the generating agent generates heat and/or singlet oxygen under the environment of a site where cancer cells are present.

The present invention is (4) the generating agent according to any of above-mentioned (1)-(3), wherein the incorporation into cells is accelerated.

The present invention is (5) the generating agent according to any of above-mentioned (1)-(4), wherein the organic peroxide is a peroxide of an imidazole derivative.

The present invention is (6) the generating agent according to any of above-mentioned (1)-(4), wherein the chemiluminescent compound is a dioxetane compound.

The present invention is (7) a pharmaceutical composition for cancer treatment, comprising an organic peroxide or chemiluminescent compound generating heat and/or singlet oxygen.

The present invention is (8) a pharmaceutical composition for inducing sudden death of cells, comprising an organic peroxide or a chemiluminescent compound generating heat and/or singlet oxygen.

The present invention is (9) a compound represented by

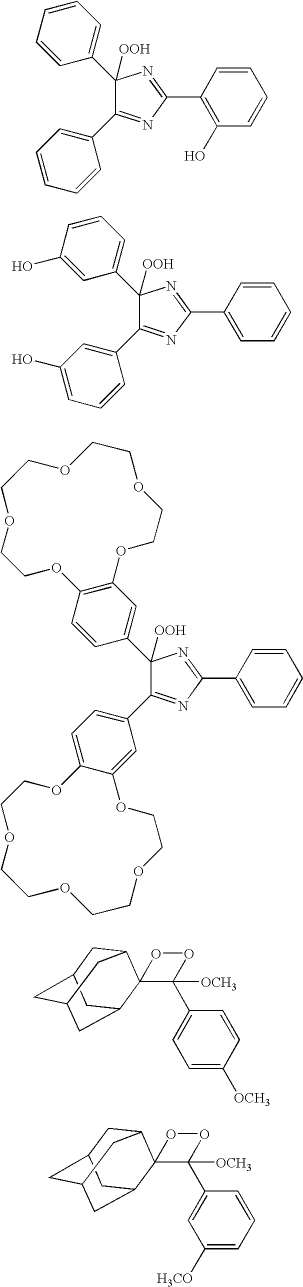

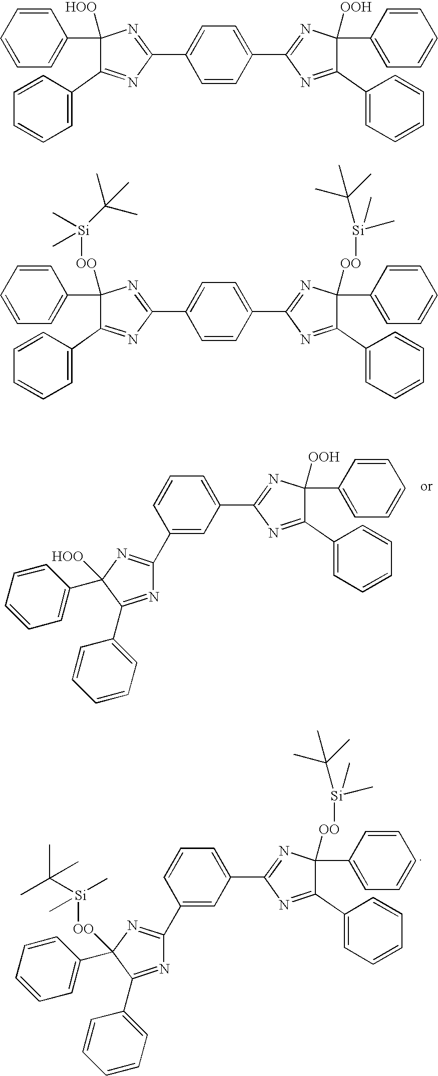

### BEST MODE FOR CARRYING OUT THE INVENTION

The organic peroxide according to the present invention includes, for example, a hydroperoxide, a percarboxylic acid, a dialkyl peroxide, a diacyl peroxide, an ester peroxide, a cyclic peroxide, an organic metal peroxide, a peroxide of an imidazole derivative, and is preferably a peroxide of an imidazole derivative.

For the peroxide of imidazole derivative, for example, 4-hydroperoxides and 4-silyl peroxides of an imidazole are particularly preferred, and the endoperoxide is also included.

As the peroxide of imidazole derivative, for example, the following compounds are mentioned:
General formula 1:

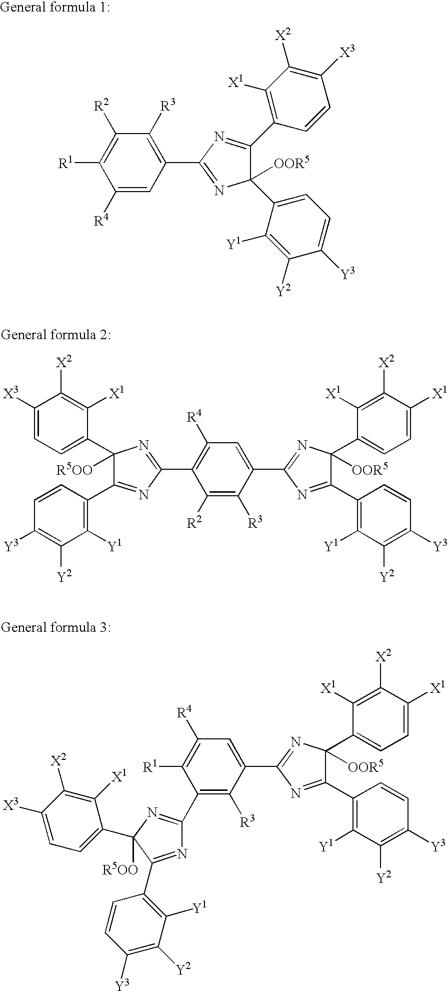

In general formulae 1, 2 and 3, R¹ to R⁴ denote a substituent group or an atomic group, and they are not limited particularly as long as they improve functions of the peroxide of imidazole as an anticancer agent. R¹ to R⁴ independently represent a hydrogen atom or an appropriate substituent group. As the substituent group as R¹ to R⁴, for example, a lower alkyl substituted amino group such as a primary amino group, a methylamino group, and a dimethylamino group, a halogen group such as a fluoro group, a chloro group, a bromine group, and an iodine group, a hydroxy group, a carboxyl group, a cyano group, a nitro group, and a formyl group are mentioned, and furthermore, in any of above-mentioned substituent group, one or more hydrogen atoms thereof may be further substituted by above-mentioned other substituent groups. The peroxide of an imidazole in which R¹ and/or R² is a hydroxyl group is preferred. In the peroxide of an imidazole in which R¹ is a lower alkyl substituted amino group, the alkyl group in the alkyl amino group may be bound to an adjacent carbon atom each other such as a carbon atom to which R² and/or R⁴ is bound, to form a ring structure such as a piperidine ring and a julolidine ring. Besides, R¹ to R⁴ may be same or different each other and a heterocyclic group or an aromatic ring group of monocyclic or condensed polycyclic type, and the heterocyclic group or the aromatic ring group may have one or more substituent groups. As the heterocyclic group of R¹ to R⁴, for example, an imidazoline ring, an imidazole ring, an oxazoline group, an oxazole ring, an isoxazole ring, a thiazoline ring, a thiazole ring, an isothiazole ring, a pyrrole ring, and a furan ring are mentioned, and as the aromatic ring group, for example, a benzene ring, a naphthalene ring, and an anthracene ring are mentioned.

In general formulae 1, 2 and 3, R⁵ represents a substituent group or an atomic group, and they are not limited particularly as long as they improve functions of the peroxide of imidazole as an anticancer agent. For example, a functional group removable under a hydrolysis condition such as a hydrogen atom, a trimethylsilyl group, a dimethyl t-butylsilyl group, a triisopropyl silyl group, and an acyl group are mentioned.

In general formulae 1, 2 and 3, X¹, X² and X³ represent a substituent group or an atomic group, and they are not limited particularly as long as they improve functions of the peroxides of imidazole as an anticancer agent. For example, a lower alkyl substituted amino group such as a primary amino group, a methylamino group and a dimethylamino group, a halogen group such as a fluoro group, a chloro group, a bromine group, and an iodine group, a hydroxy group, a carboxy group, a cyano group, and a nitro group are mentioned, and furthermore, in any of above-mentioned substituent groups, one or more hydrogen atoms thereof may be further substituted by above-mentioned other substituent groups.

In general formulae 1, 2 and 3, Y¹, Y² and Y³ represent a substituent group or an atomic group, and they are not limited particularly as long as they improve functions of the peroxide of imidazole as an anticancer agent. For example, a lower alkyl substituted amino group such as a primary amino group, a methylamino group, and a dimethylamino group, a halogen group such as a fluoro group, a chloro group, a bromine group, and an iodine group, a hydroxy group, a carboxy group, a cyano group, and a nitro group are mentioned, and furthermore, in any of above-mentioned substituent groups, one or more hydrogen atoms thereof may be further substituted by above-mentioned other substituent groups.

Compounds of general formulae 1, 2 and 3 according to the present invention generate heat and singlet oxygen.

As the chemiluminescent compound according to the present invention, compounds of firefly luciferin, Vargula luciferin, luminol, acridine, lucigenin and dioxetane compounds are mentioned, and the dioxetane compound is preferred.

As the dioxetane compound, for example, compounds of tetraalkyl dioxetane, dioxetanone, and dioxetadione are mentioned.

As the preferred dioxetane compound, 3-(2′-spiroadamantane)-4-methoxy-4-(4″-methoxy)phenyl-1,2-dioxetane represented by 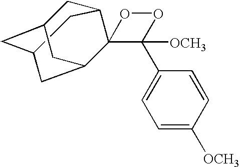
and 3-(2′-spiroadamantane)-4-methoxy-4-(3″-methoxy)phenyl-1,2-dioxetane represented by 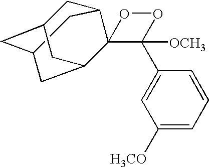
are mentioned.

The chemiluminescent compound according to the present invention generates heat.

In the present invention, a heat generating agent or an anticancer agent comprising an imidazole peroxide derivative or dioxetane compound generates a reaction heat of approximately 20 Kcal/mol to 90 Kcal/mol. Meanwhile, singlet oxygen is generated at a yield of approximately upto 50%. The MTT antitumor sensitivity test in which a cell line of large intestine tumor was incubated with an imidazole peroxide derivative for 48 hours revealed that the derivative exhibited sharply effects at 50-100 μM/cm² and showed high level of antitumor performance same as a commercially available MMC to give a survival rate of 11%.

Compounds formed from the 4-hydroperoxide and the 4-silyl peroxide of imidazoles are corresponding amidines, imidazoles, and, in some cases, singlet oxygen having cell activity, which are normally considered to be nontoxic. Further, compounds formed from dioxetanes are corresponding ketones. Therefore, the heat generating agent and the pharmaceutical composition according to the present invention can minimize side effects caused by deactivation or death of normal cells. Moreover, these have the feature of inducing sudden death of cancer cells. The cancers, which are curable by the present invention, include, but are not particularly limited to, liver cancer, lung cancer, stomach cancer, large intestine cancer, skin cancer and uterine cancer.

In the present invention, in order to deliver the heat generating agent or the pharmaceutical composition to cancer cells, they are applied to an affected area or normally injected. In addition, for example, a medical catheter is inserted from an inguinal region or the like, passed through blood vessels to target a cancer site, and then the heat generating agent or the pharmaceutical composition in solution is transferred through the catheter. Alternatively, they may be directly delivered to the lesion using a syringe, and in this case, they are preferably delivered in such a way that the cancer cells may be killed as quickly as possible.

In the present invention, products formed by degradation of the organic peroxide or the chemiluminescent compound preferably have easily metabolizable structures. The peroxide of imidazole derivative or its endoperoxide or the dioxetane compound in the present invention is preferable to use, because it produces the corresponding imidazole, amidine or ketone which is diffused in a living body to give little influence on normal cells.

Further, in order to enhance degradation of the organic peroxide such as the peroxide of imidazole derivative or the chemiluminescent compound such as the dioxetane compound, an aqueous solution of KOH or NaOH, an organic base amine or an inorganic base containing F⁻ can be injected to an affected portion to accelerate the reaction.

It is preferable that the peroxide of imidazole, its endoperoxide or the dioxetane compound is decomposed to give a product which has a easily metabolizable structure, and that a small amount of the peroxide is administered.

The peroxide of an imidazole derivative represented by general formula (1) in the present invention can be synthesized by the method of, for example, reaction formula 1, reaction formula 2 and reaction formula 3 (and reaction formula 4) shown below.

Synthesis Method 1 (Synthesis of Benzils)

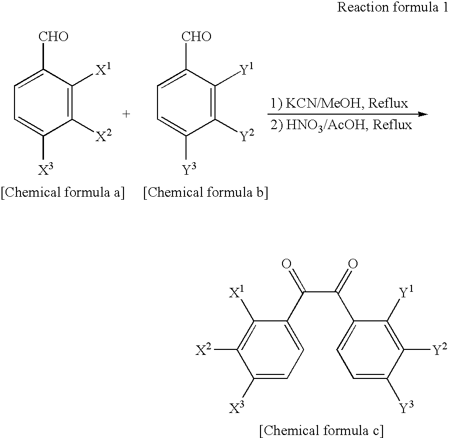

Synthesis Method 2 (Synthesis of Imidazoles)

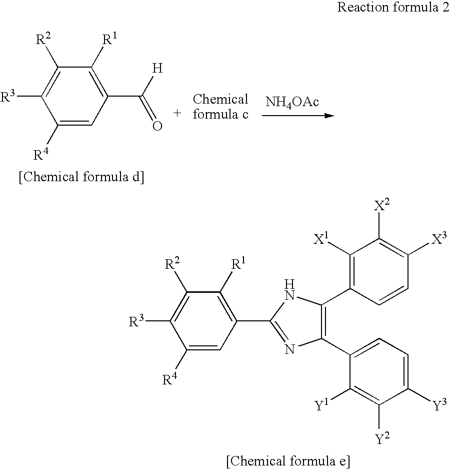

Synthesis Method 3 (Synthesis of Peroxides)

Reaction is carried out in accordance with reaction formula (3) by the method of White et al. to synthesize the peroxide of an imidazole derivative represented by general formula (1), including the peroxide wherein X and Y are, in general, different substituent groups.

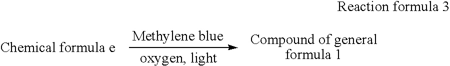

Synthesis Method 4 (Synthesis of Alkylsilyl Derivatives)

Trialkylsilyl derivatives of the peroxide represented by general formula (1) can be synthesized by reaction in accordance with reaction formula 4. 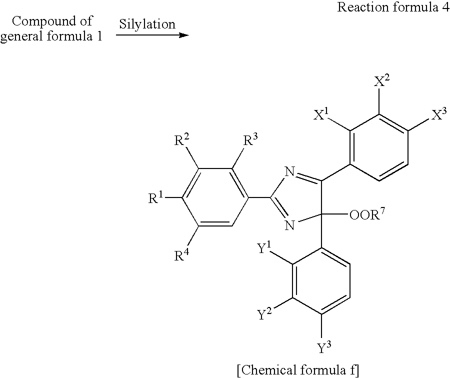
(In the chemical formula, R⁷ denotes a trialkylsilyl group, and the alkyl group is a straight chain or branched chain alkyl group of C₁-C₆, particularly C₁-C₄.)

Imidazole derivatives represented by general formula (2) or (3) in the present invention and the silylation product thereof can be synthesized using the compound represented by 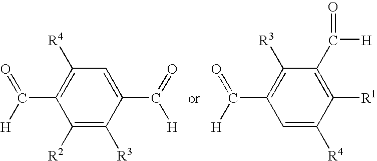
in the place of the compound of [Chemical formula d] in the synthesis method 2 of the imidazole derivative represented by general formula (1).

### EXAMPLES

The present invention will be explained hereafter by, but is not limited to, Examples and Test examples.

### Example 1

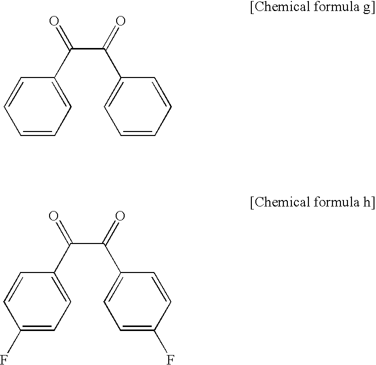

Benzils to use as the raw materials were synthesized according to reaction formula 1 by benzoin condensation of corresponding benzaldehyde followed by nitric acid oxidation. Contrastive [Chemical formula g] was synthesized by the method of Davidson et al. (Davidson, D.; Weiss, M.; Jelling. J. Org. Chem. 1937, 2, 319) and [Chemical formula h] was synthesized by the method of Luts et al. (Luts, R. E.; Murphey, R. S. J. Am. Chem. Soc, 1949, 71, 478).

### Example 2

Imidazole derivatives were synthesized in accordance with Reaction formula 2 using the method of Davidson et al. (Davidson, D.; Weiss, M.; Jelling. J. Org. Chem. 1937, 2, 319). [Chemical formula g] or [Chemical formula h] was refluxed with one equivalent or a slightly excessive amount of the corresponding substituted benzaldehyde and 10 equivalents of ammonium acetate in acetic acid for 4-5 hours, processed by a conventional method, and purified by recrystallization to get the corresponding imidazoles (compounds shown below) at their reasonable yields (50-80%). Their respective spectral data and the like are shown below.

2,4,5-Triphenyl imidazole (D. Davidson, M. Weiss, and M. Jelling, J. Org. Chem., 1937, 2, 319): colorless needle; mp 282.5-283° C.; IR(KBr) 1613(C═N) cm⁻¹; ¹H NMR(500 MHz, CDCl₃) δ7.13-7.60(m, 13H), 8.08(d, J=8.3 Hz, 2H), 12.7(s, 1H); UV-vis λₘₐₓ(EtOH) 303(log ε 4.42)nm; MS(FAB) m/z 297(M⁺+1); HRMS(FAB) Calcd for C₂₁H₁₇N₂ 297.1392. Found 297.1424. Anal. Calcd for C₂₁H₁₆N₂: C, 85.11; H, 5.44; N, 9.45. Found: C, 85.08; H, 5.48; N, 9.43.

4,5-Bis(4-fluorophenyl)-2-(4-dimethylaminophenyl)imidazole: colorless needle; mp 232-233° C.; IR(KBr) 1620(C═N)cm⁻¹; ¹H NMR(500 MHz, CDCl₃) δ3.01(s, 6H), 6.71(br s, 2H), 7.00(br s, 4H), 7.45(br s, 4H), 7.83(br s, 2H); UV-vis λₘₐₓ(CH₂Cl₂) 230(log ε 4.1), 323(4.5) nm; Anal. Calcd for C₂₃H₁₉F₂N₃: C, 73.58; H, 5.10; N, 11.19. Found.

2-(4-Hydroxyphenyl)-4,5-diphenylimidazole [A. H. Cook, D. G. Jones; J. Che. Soc., 278(1941)]: colorless needle; mp 273-275° C.; ¹H NMR(200 MHz, DMSO-d₆) δ 6.84(d, J=8.4 Hz, 2H), 7.20-7.59(m, 10H), 7.88(d, J=8.4 Hz, 2H), 9.70(s, 1H), 12.4(s, 1H); IR(KBr) υₘₐₓ 3162(O—H), 1613(C═N), 1493, 1466, 1396, 1224, 1180, 839, 766, 739, 698 cm⁻¹; UV-vis(EtOH); λₘₐₓ 221(log ε=4.27), 298(4.43) nm; HRMS(FAB) Calcd for C₂₁H₁₇N₂O 313.1341(M+H⁺). Found 313.1341. Anal. Calcd for C₂₁H₁₆N₂O.H₂O: C, 76.34; H, 5.49; N, 8.48. Found: C, 76.46; H, 5.69; N, 8.23.

2-(3-Hydroxyphenyl)-4,5-diphenylimidazole(F. R. Japp, H. H. Robinson; Chem. Ber., 15, 1269(1882): colorless plate; mp 273-275° C.; ¹H NMR(200 MHz, DMSO-d₆) δ 6.77(d, J=7.2 Hz, 1H), 7.16-7.58(m, 13H), 9.55(s, 1H), 12.6(s, 1H); IR(KBr) υₘₐₓ 3380(O—H), 1593(C═N), 1483, 1448, 1400, 1352, 1230, 1193, 791, 764, 729, 696 cm⁻¹; UV-vis(EtOH) λₘₐₓ 222(log ε=4.43), 304(4.43) nm; HRMS(FAB) Calcd for C₂₁H₁₇N₂O 313.1341(M+H⁺). Found 313.1342. Anal. Calcd for C₂₁H₁₆N₂O: C, 80.75; H, 5.16; N, 8.97. Found: C, 80.65; H, 5.19; N, 8.92.

2-(4-Aminophenyl)-4,5-diphenylimidazole(Kallel&Co. Akt.-Ges. Ger., 1956, 950, 618): colorless needle; mp 253-256° C. (literature value 180° C.); IR(KBr)3360(N—H), 1613(C═N)cm⁻¹; ¹H NMR(500 MHz, CDCl₃) δ5.23(s, 2H), 6.48(d, J=8.5 Hz, 2H), 7.25(m, 10H), 7.60(d, J=8.5 Hz, 2H), 12.4(br s, 1H); UV-vis λₘₐₓ(EtOH)309(log ε 450) nm; MS(FAB) m/z 312(M⁺+1; 100%); HRMS(FAB) Calcd for C₂₁H₁₈N₃ 312.1501. Found 312.1483. Anal. Calcd for C₂₁H₁₇N₃.2/3H₂O: C, 77.99; H, 5.71; N, 12.99. Found: C, 77.77; H, 5.73; N, 13.01.

2-(4-Nitrophenyl)-4,5-diphenylimidazole: yellow needle; mp 255-257° C.; IR(KBr) 1603(C═N), 1518(NO₂), 1342(NO₂)cm⁻¹; ¹H NMR(500 MHz, CDCl₃) δ7.35(m, 10H), 8.09(d, J=8.4 Hz, 2H), 8.32(d, J =8.4 Hz, 2H), 9.60(s, 1H); UV-vis λₘₐₓ(EtOH)222(log ε4.28), 255(420), 386(4.29) nm; MS(FAB) m/z 342(M⁺+1; 100%); Anal. Calcd for C₂₁H₁₅N₃O₂: C, 73.89; H, 4.43; N, 12.31. Found: C, 73.87; H, 4.48; N, 12.24.

2-(4-Formylphenyl)-4,5-diphenylimidazole(B. Radziszewskii, Ber., 1877, 10, 70): yellow needle; mp244-245.5° C.; IR(KBr) 2970(C—H), 1698(C=0), 1607(C═N), 837, 766, 696 cm⁻¹; ¹H NMR(200 MHz, CDCl₃) δ7.19-7.68(m, 10H), 7.97(d, J=8.4 Hz, 2H), 8.10(d, J=8.4 Hz, 2H), 10.05(s, 1H); UV-vis λₘₐₓ(EtOH) 243(log ε 4.14), 301(3.94), 359(4.24) nm; MS(FAB) m/z 325(M⁺+1); HRMS(FAB) C₂₂H₁₆N₂O 325.1341. Found 325.1311. Calcd for Anal. Calcd for C₂₂H₁₆N₂O: C, 81.46; H, 497; N, 8.64. Found: C, 81.21; H, 5.02; N, 8.58.

2-(2′,4′,6′-Trimethylphenyl)-4,5-diphenylimidazole [G. R. Coraor, L. A. Cescon, R. Dessauer, E. F. Silversmith and E. J. Urban J. Org. Chem., 1971, 36(16), 2262-2267]: colorless needle; mp 242-243° C.; IR(KBr) 2922(C—H), 1605(C═N)cm⁻¹; ¹H NMR(500 MHz, CDCl₃) δ2.23(s, 6H), 2.32(s,3H), 6.93(s, 2H), 7.32(br s, 6H), 7.47(br s, 2H), 7.69(br s, 2H), 8.81(br s, 1H); UV-vis λₘₐₓ(EtOH) 222(log ε 439), 284(4.17) nm; MS(FAB)m/z 339(M⁺+1); HRMS(FAB) Calcd for C₂₄H₂₃N₂ 339.1861. Found 339.1860. Anal. Calcd for C₂₄H₂₃N₂ 1/2H₂O: C, 82.96; H, 6.67; N, 8.06. Found: C, 83.07; H, 6.86; N, 7.93.

### Example 3

The peroxide of an imidazole derivative was synthesized according to reaction formula (3) by the method of White et al. (E H. White and M. J. C. Harding, Photochem. Photobiol., 1965, 4, 1129-1155).

Various imidazole derivatives obtained in Example 2 were dissolved into dichloromethane at −78° C., added with a few drops of methylene blue as a sensitizer, and irradiated with artificial daylight while blowing oxygen for 4-6 hours. Upon completion of the reaction, the reaction mixtures were immediately added with alcohol, and subjected to evaporation of dichloromethane at a low-temperature (15° C. or lower) to separate. Crystals thus obtained were washed with alcohol to get the peroxides (Chemical formula A-Chemical formula H, and Chemical formula J-Chemical formula L) shown below at high purities and high yields. Further, the peroxide similarly obtained was subjected to silylation by the following method to get silylated peroxide (Chemical formula I).

(Silylation method): The method of Corey et al. was used for silylation of peroxides (E. J. Corey and A. Venkateswaru, J. Am. Chem. Soc., 1972, 94, 6190-6191. G. R. Clark, M. M. Nikaido, C. K. Fair and J. Lin, J. Org. Chem., 1985, 50, 1994-1996). Namely, the peroxide was added with 5 equivalents of tert-butyldimethylsilyl chloride and a catalytic amount of pyridine, and subjected to chromatography on silica gel to separate and purify.

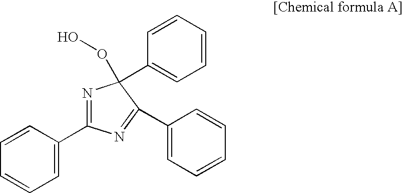

4-Hydroperoxy-2,4,5-triphenyl-4H-isoimidazole (E. H. White and M. J. C. Harding, Photochem. Photobiol., 1965, 4, 1129-1155): colorless powder; mp 108-110° C. (dec.) (lit. ¹¹), 110° C.); IR(KBr) 1613(C═N) cm⁻¹; ¹H NMR(500 MHz, CDCl₃) δ7.22(t, J=7.8 Hz, 2H), 7.30-7.37(m, 4H), 7.48(dd, J=7.5, 5.5 Hz, 2H), 7.52(t, J=7.5 Hz, 2H), 7.59(t, J=7.5 Hz, 1H), 7.95(d, J=7.5 Hz, 2H), 8.38(d, J=7.5 Hz, 2H), 13.62(br s, 1H); ¹³C NMR(67 MHz, DMSO-d₆) δ107.3(s), 124.5(d), 128.0(d), 128.6(d), 128.7(d), 128.9(d), 129.1(d), 129.5(d), 129.6(d), 131.3(s), 132.1(d), 132.8(d), 137.9(s), 169.6(s), 193.9(s); UV-vis λₘₐₓ(EtOH) 228(log ε 4.25), 281(4.32) nm; MS(FAB) m/z 329(M⁺+1); HRMS(FAB) Calcd for C₂₁H₁₇N₂O₂ 329.1265. Found 329.1290. Anal. Calcd for C₂₁H₁₇N₂O₂: C, 76.81; H, 4.91; N, 8.53. Found: C, 76.45; H, 4.94; N, 8.43.

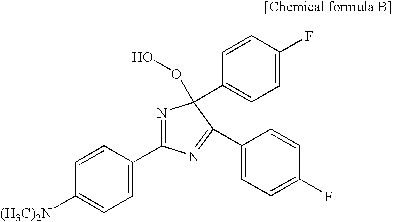

4,5-Bis(4-fluorophenyl)-4-hydroperoxy-2-(4-dimethylaminophenyl)-4H-isoimidazole (M. Kimura. H. Nishikawa, H. Kura., H. Lim, and E. H. White, CHEMISTRY LETTERS, 1993, 505-508): orange powder; mp 125-128° C. (dec.); IR(KBr) 1603(C═N) cm⁻¹; ¹H NMR(500 MHz, CDCl₃) δ3.01(s, 6H), 6.42(d, J=8.9 Hz, 2H), 6.99(t, J=8.8 Hz, 2H), 7.17(t, J=8.8 Hz, 2H), 7.44(dd, J=8.8, 5.3 Hz, 2H), 7.88(d, J=8.9 Hz, 2H), 8.35(dd, J=8.8, 5.3 Hz, 2H), 12.82(br s, 1H); UV-vis λₘₐₓ (CH₂C1₂) 229(log ε 4.1), 307(4.2), 402(4.1) nm; Anal. Calcd for C₂₃H₁₉F₂N₃O₂: C, 67.81; H, 4.70; N, 10.31. Found: C, 67.35; H, 4.66; N, 10.12.

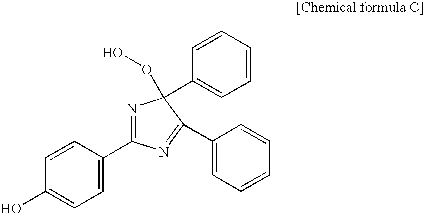

4-Hydroperoxy-2-(4-hydroxyphenyl)-4,5-diphenyl-4H-imidazole: 288 mg of the raw material was irradiated with artificial daylight under bubbling oxygen for three hours to obtain it a pale yellow crystal (241 mg, 76%). mp 125-127° C. (dec.); ¹H NMR(300 MHz, DMSO-d₆) δ6.93(d, J=8.5 Hz, 2H), 7.20-7.63(m, 8H), 8.08(d, J=7.3 Hz, 2H), 8.17(d, J=8.5 Hz, 2H), 10.2(s,1H), 12.2(br s, 1H); IR(KBr),υₘₐₓ 3396(O—H), 1607(C═N), 1510, 1437, 1319, 1278, 1170, 1087, 849, 754, 681 cm⁻¹; UV-vis(DMSO) λₘₐₓ 295(log ε=4.29)nm; HRMS(FAB) Calcd for C₂₁H₁₇N₂O₃ 345.1239(M+H⁺). Found 345.1252. Anal. Calcd for C₂₁H₁₆N₂O₃.1/2H₂O: C, 71.38; H, 4.85; N, 7.93. Found: C, 71.19; H, 4.88; N, 7.72.

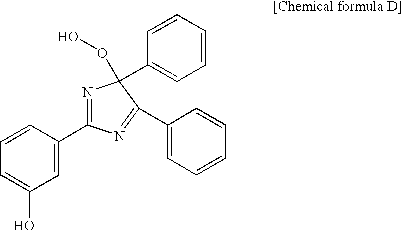

This compound was obtained as a pale yellow crystal (245 mg, 72%) by irradiating 399 mg of the raw material with artificial daylight under bubbling oxygen for three hours.

mp 111-113° C.(dec.); ¹H NMR(300 MHz, CDCl₃) δ6.87(ddd, J=7.8, 2.6, 1 Hz, 1H), 7.13(t, J=7.8 Hz, 1H), 7.29-7.63(m, 10H), 8.33(m, J=7.7 Hz, 2H), 13.7(s,1H); IR(KBr)υₘₐₓ 3360(O—H), 1613(C═N), 1508, 1450, 1284, 780, 758, 743, 689 cm⁻¹; UV-vis(CH₂Cl₂) λₘₐₓ 288(log ε=4.29)nm; HRMS(FAB) Calcd for C₂₁H₁₇N₂O₃ 345.1239(M+H⁺). Found 345.1207. Anal. Calcd for C₂₁H₁₆N₂O₃.1/2H₂O: C, 71.38; H, 4.85; N, 7.93. Found: C, 71.38; H, 4.87; N, 7.76.

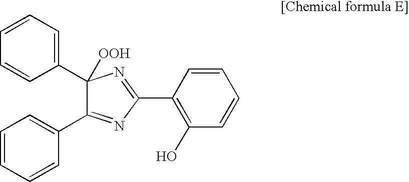

### 4-Hydroperoxy-2-(2-hydroxyphenyl)-4,5-diphenyl-4H-isoimidazole

2-(2-Hydroxyphenyl)-4,5-diphenyl imidazole (420 mg, 1.34 mmol) in CH₂Cl₂ (60 ml) and a catalytic amount of methylene blue in MeOH (1 ml) were irradiated with artificial daylight under an O₂ atmosphere at −78° C. for seven hours. The reaction was monitored by TLC. Upon completion of the reaction, the sensitizer was removed by silica gel syringe column chromatography (CH₂Cl₂). The catalyst was concentrated under a reduced pressure and dried. The title compound was obtained as a purple crystal (346 mg, 75%).

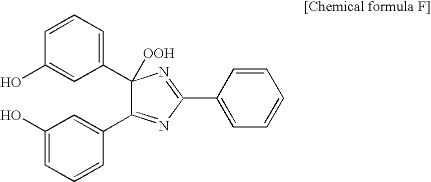

### 4-Hydroperoxy-4,5-bis(3-hydroxyphenyl)-2-phenyl-4H-isoimidazole

4,5-Bis(3-hydroxyphenyl)-2-phenylimidazole (100 mg, 0.305 mmol) in CH₂Cl₂ and MeOH and an adduct polymer rose bengal (500 mg) were irradiated with artificial daylight under an O₂ atmosphere at −78° C. for three hours. The reaction was monitored by TLC. Upon completion of the reaction, the sensitizer was removed by filtration. The catalyst was concentrated under a reduced pressure and the residues were dried. The title compound was obtained as a colorless crystal (95 mg, 86%).

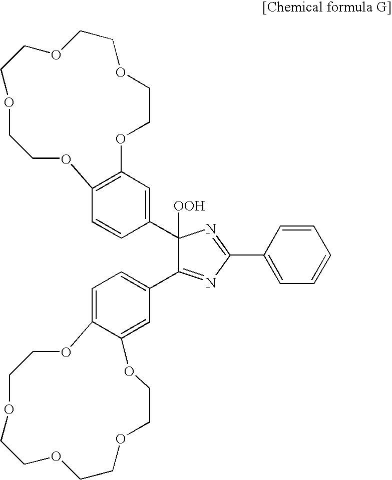

Bis(crown ether)iophineperoxide: yellow crystal; mp 99-101° C.; ¹H NMR(500 MHz, CDCl₃) δ13.61(bs), 8.00(m, 2H), 7.96(d, 1H, J=2.0 Hz), 7.89(dd, 1H, J=2.0 Hz, 8.5 Hz), 7.40(bs, 1H), 7.35-7.30(m, 1H), 7.24-7.20(m, 2H), 6.91(d, 1H, J=8.5 Hz), 6.74(dd, 1H, J=2.0 Hz, 8.5 Hz), 6.69(d, 1H, J=8.5 Hz), 4.30-4.20 (m, 4H), 4.15-4.05(m, 4H), 3.97-3.92(m, 4H), 3.86(t, 2H, J=4.5 Hz), 3.82(t, 2H, J=4.5 Hz), 3.80-3.75(m, 8H), 3.75-3.69(m, 8H)

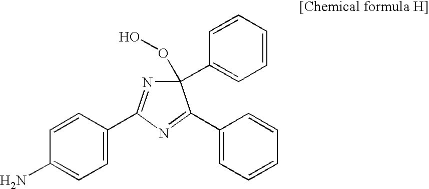

2-(4-Aminophenyl)-4-hydroperoxy-4,5-diphenyl-4H-isoimidazole

(E. Vedejs, and P. L. Fuchs, J. Org. Chem., 1971, 36, 366-367.): yellow powder; mp 147-149° C. (dec.); IR(KBr) 3376(N—H), 1603(C═N), 762, 692 cm⁻¹; ¹H NMR(500 MHz, CDCl₃) δ4.02(br s, 2H), 6.54(d, J=9.0 Hz, 2H), 7.29(m, 3H), 7.42-7.49(m, 4H), 7.55(t, J=7.5 Hz, 1H), 7.93(d, J=9.0 Hz, 2H), 8.32(d, J=7.5 Hz, 2H); UV-visλₘₐₓ (EtOH) 209(log ε 4.33), 300(4.20), 376(3.95) nm; MS(FAB) m/z 344(M⁺+1)

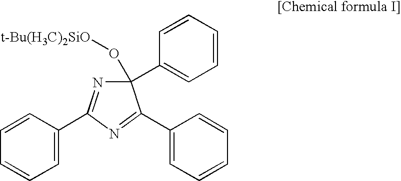

### 4-t-Butyldimethylsilylperoxy-2,4,5-triphenyl-4H-isoimidazole

colorless powder; mp 93.5-96.0° C.; IR(KBr) 2960(C—H), 1618(C═N), 886, 826(Si—O)cm⁻¹; ¹H NMR(500 MHz, CDCl₃) δ0.145(s, 3H), 0.197(s, 3H), 0.843(s, 9H), 7.25-7.29(m, 3H), 7.30-7.35(m, 2H), 7.43(t, J=8.0 Hz, 2H), 7.49-7.58(m, 4H), 8.22(d, J=8.0 Hz, 2H), 8.48(d, J=7.0 Hz, 2 Hz); UV-visλₘₐₓ(CH₂Cl₂) 232(log ε 4.22), 243(4.20), 279(4.30) nm; MS(FAB) m/z 443(M⁺+1); HRMS(FAB) Calcd for C₂₇H₃₁N₂O₂Si 443.2155. Found 443.2139. Anal. Calcd for C₂₇H₃₀N₂O₂Si.1/2H₂O: C, 71.80; H, 6.92; N, 6.20. Found: C, 72.06; H, 6.80; N, 6.17.

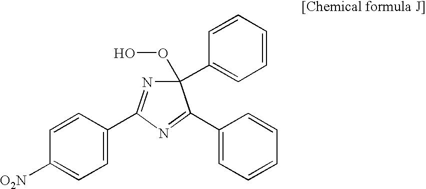

pale yellow powder; mp 148-159° C. (dec.); IR(KBr) 1524(NO₂), 1350(NO₂) cm⁻¹; ¹H NMR(500 MHz, CDCl₃) δ7.32-7.38(m, 3H), 7.43(dd, J=8.4, 2.0 Hz, 2H), 7.55(t, J=8.1 Hz, 2H), 7.65(t, J=8.1 Hz, 1H), 8.08(d, J=9.2 Hz, 2H), 8.18(d, J=9.2 Hz, 2H), 8.37(d, J=8.1 Hz, 2H), 12.9(s, 1H); UV-visλₘₐₓ(EtOH) (log ε) nm; MS(FAB) m/z 374(M⁺+1); Anal. Calcd for C₂₁H₁₅N₃O₄. 1/4H₂O: C, 66.75; H, 4.13; N, 11.12. Found: C, 66.73; H, 4.00; N, 11.13.

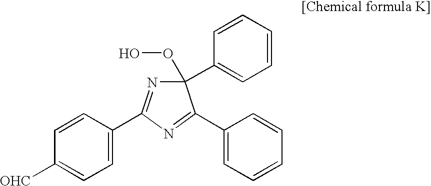

### 2-(4-Formylphenyl)-4-hydroperoxy-4,5-diphenyl-4H-isoimidazole

[M. Kimura, M. Tsunenaga, T. Koyama, H. Iga, R. Aizawa, Y. Tachi, and Y. Naruta, ITE Letters on Batteries, New Technologies & Medicine, 1, C8 30-34(2002)]: pale yellow powder; mp 97.0-98.5° C. (dec.); IR(KBr) 1705(C═O), 1607(C═N), 835, 690 cm⁻¹; ¹H NMR(500 MHz, CDCl₃) δ7.31-7.38(m, 3H), 7.45(m, 2H), 7.53(t, J=7.5 Hz, 2H), 7.63(t, J=7.5 Hz, 1H), 7.75(d, J=8.0 Hz, 2H), 8.18(d, J=8.0 Hz, 2H), 8.36(d, J=7.5 Hz, 2H), 10.02(s, 1H), 12.69(br s, 1H); UV-visλₘₐₓ (EtOH) 281(log ε 4.48) nm; HRMS(FAB) Calcd for C₂₂H₁₇N₂O₃ 357.1239. Found 357.1216. Anal. Calcd for C₂₂H₁₆N₂O₃: C, 74.15; H, 4.53; N, 7.86. Found: C, 74.29; H, 4.62; N, 9.44.

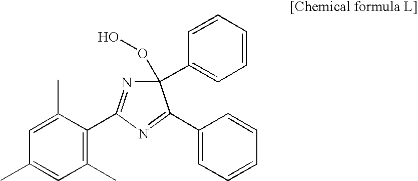

### 4-Hydroperoxy-2-(2′,4′,6′-trimethylphenyl)-4,5-diphenyl-4H-isoimidazole

[M. Kimura, M. Morioka, M. Tsunenaga, and Z-Z Hu, ITE Letters on Batteries, New Technologies & Medicine, 1, C25 418-421(2002)]: colorless powder; mp 157-158.5° C. (dec). (lit, 158-159.5° C.); IR(KBr) 2922(C—H), 1615(C═N) cm⁻¹; ¹H NMR(200 MHz, CDCl₃) δ1.97(s, 6H), 2.31(s, 3H), 6.83(s, 2H), 7.35-7.57(m, 8H), 8.21(d, J=7.6 Hz, 2H), 12.40(br s, 1H); UV-visλₘₐₓ (CH₂Cl₂) 229(log ε 4.07) 297(4.18) nm; Anal. Calcd for C₂₄H₂₂N₂O₂: C, 77.81; H, 5.99; N, 7.56. Found: C, 77.64. H, 6.07; N, 7.57.

### Test Example 1

[Anticancer Effect I]

Cytotoxicity was determined as shown below using MTT method proposed by Mosmann et al. (Mosmann, T.; Rapid colorimetric assay for cellular growth and survival: application proliferation and cytotoxicity assays. J. Immunol. Meth. 65: 55-63, 1983). An established cell line from human large intestine cancer was adjusted on a 10% FCS-containing RPMI 1640 culture solution to have 5×10³ cells/100 ml, plated in a 96-well microplate, and incubated for 48 hours. Then, the resultant was added with 100 ml of a peroxide, and incubated under a 5% CO₂ condition at 37° C. for 48 hours to determine cytotoxicity by MTT assay¹. MTT assay: Upon completion of the incubation, 20 ml of MTT reagent (5 mg/ml in PBS) was added to each well, and then the formazan left on the bottom of the plate was added with 0.04N HCl to dissolve in isopropanol. OD was measured at a test wavelength and at a reference wavelength of 630 nm.

Survival rate in percentage was calculated by the following equation:
Survival rate=(OD test value/OD reference value)×100 (%)

Results obtained for commercially available mitomycin C (MMC) are also shown in Table 1 for comparison.

TABLE 1

| | Concentration of | Survival rate of |
|---|---|---|
| | anticancer agent | cancer cells |
| Anticancer agent | (μM/ml) | % |
| Chemical formula A | 1 | 100.1 |
| | 10 | 100.1 |
| | 100 | 78.3 |
| Chemical formula B | 1 | 100.3 |
| | 10 | 93.7 |
| | 100 | 37.0 |
| Chemical formula C | 1 | 91.9 |
| | 10 | 82.2 |
| | 100 | 11.1 |
| Chemical formula D | 1 | 93.3 |
| | 10 | 92.2 |
| | 100 | 71.2 |
| Chemical formula E | 1 | — |
| | 10 | 88.1 |
| | 100 | 11.8 |
| Chemical formula F | 1 | — |
| | 10 | 87.4 |
| | 100 | 42.2 |
| Chemical formula G | 1 | — |
| | 10 | 87.2 |
| | 100 | 48.2 |
| Chemical formula H | 1 | 100.0 |
| | 10 | 97.5 |
| | 100 | 87.4 |
| Chemical formula I | 1 | 97.2 |
| | 10 | 95.9 |
| | 100 | 60.6 |
| MMC | 1 | 65.0 |
| | 10 | 28.0 |
| | 100 | 14.5 |

As is evident from Table 1, Chemical formula C, Chemical formula E and the like of the present invention are comparable to the commercially available MMC in anticancer effect at a concentration of 100 μM/L. The results demonstrate that these peroxides are effective as anticancer agents.

### Test Example 2

[Measurement I]

Reaction heat and chemiluminescent efficiency relating to chemiluminescent reaction of the peroxides (Chemical formula A to Chemical formula L) were measured. Further, the amount of imidazole formed that indicates generation efficiency of singlet oxygen generated by this reaction was measured. Results thus obtained are summarized in Table 2.

Measurement of reaction heat: heat generated by above-mentioned compounds was measured by a differential thermal analyzer.

Heat measurement of a chemiluminescent system peroxide in the solid state was conducted using a differential thermal analyzer as follows: 2 to 3 mg of the peroxide was weighed, filled into an aluminum capsule, and heated gradually to 80-180° C. by DSC-50 (Shimadzu Corporation) to measure the generated heat.

Measurement of relative light intensity of chemiluminescence: Reaction heat of the peroxides (Chemical formula A to Chemical formula L) in the solid state was measured and light intensity of the peroxides in the methanol solution mixed with 1N KOH methanol solution at a ratio of 10:1 was measured by PMA apparatus (Hamamatsu Photonics), and apparatus light intensity was determined while the luminescence of Chemical formula A was defined to be 1 for reference. Results are summarized in Table 2.

Formation of imidazole: the reaction solution was subjected to liquid chromatography to determine it: Developing phase Sephadex; Developer=Water:Ethanol (1:1). Results are summarized in Table 2.

TABLE 2

| | Solution | | Solid | Solid | Relative |
|---|---|---|---|---|---|
| | Reaction | Yield of | reaction | reaction | amount of |
| | heat^{a}/ | imidazole^{a} | heat/ | imidazole | chemilumi- |
| Entry | kcal/mol | % | kcal/mol | yield % | nescence^{b} |
| Chemical | 53.8 | 13 | 18.7 | 45 | 1 |
| formula A | | | | | |
| Chemical | 66.3 | ˜0 | 61.0 | ˜0 | 160 |
| formula B | | | | | |
| Chemical | —^{c} | —^{c} | 53.6 | —^{c} | 1.02 |
| formula C | | | | | |
| Chemical | —^{c} | —^{c} | 47.6 | —^{c} | 0.232 |
| formula D | | | | | |
| Chemical | 46.0 | 3 | 35.3 | ˜0 | 1.6 |
| formula H | | | | | |
| Chemical | —^{c} | —^{c} | 91.4 | —^{c} | 0.58 |
| formula I | | | | | |
| Chemical | 48.7 | 49 | 52.3 | 50 | 0.60 |
| formula J | | | | | |
| Chemical | 22.4 | 58 | 15.0 | 55 | ˜0 |
| formula K | | | | | |
| Chemical | 54.0 | ˜0 | 50.5 | ˜0 | 2.2 |
| formula L | | | | | |

^{a}Reaction was started with 1N KOH/MeOH
^{b}Relative luminescence efficiency while Chemical formula A is defined to be 1 for reference.
^{c}No measurement.

### Test Example 3

[Reaction Example]

The peroxide represented by general formula (1) takes a chemiluminescent reaction and a reaction in an alcohol 10 solvent in accordance with reaction formula (5) shown below. Singlet oxygen forms a pair with formation of imidazole (Chemical formula e), and generation of heat forms a pair with formation of amidine (Chemical formula i).

[Confirmation of Singlet Oxygen]

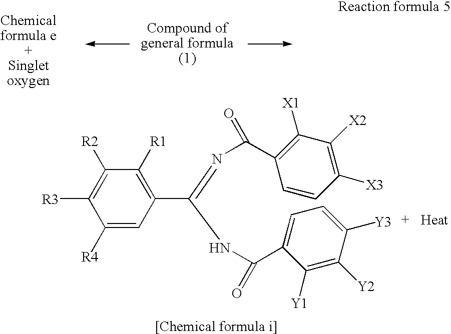

Constituents of a product given under a condition for chemiluminescent reaction were identified by HPLC. Measurement conditions: Column Intersil ODS-3 (46 mm×150 mm); Solvent MeOH:H₂O=7:3; rate 1.0 ml/min; reaction conditions: peroxide concentration: (5×10⁻³ M/CHCl₃) 1.0 ml, base concentration: 0.5 M KOH/MeOH 0.10 ml, reaction time: Left to stand for 10 min. after mixing, and neutralized by acetic acid. Singlet oxygen was confirmed by an infrared spectrometer (Tohoku Electronic) and determined quantitatively with 1,3-diphenyl benzofuranbenzo. The corresponding imidazole [Chemical formula e] and singlet oxygen were formed in an equivalent amount. The amount of [Chemical formula e] formed can be determined to give the accurate amount of formed singlet oxygen. Although it is considered that the decomposition product is composed of [Chemical formula e] and the amidine [Chemical formula i], the amidine is easily hydrolyzed and hence could not be determined directly. It has been revealed that [Chemical formula A], [Chemical formula J] and [Chemical formula K] are particularly good singlet oxygen generators (Table 2).

### Example 4

[Synthesis of Dioxetanes]

[Chemical formula j] and [Chemical formula k] shown below were synthesized by the method of E. F. Ullman et al. (U.S. Pat. No. 3,689,391 (1972)), and changed to the dioxetanes ([Chemical formula M] and [Chemical formula N]) of the present invention by reaction formula 6 or 7 shown below in accordance with peroxidation of an imidazole derivative.

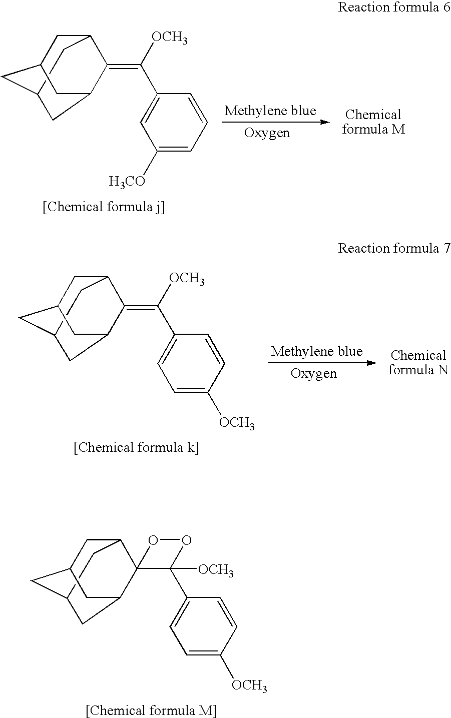

### 3-(2′-Spiroadamantane)-4-methoxy-4-(4″-methoxy)phenyl-1,2-dioxetane

¹H NMR(500 MHz, CDCl₃) 0.97(d, J=12.0 Hz, 1H), 1.23(d, J=13.3 Hz, 1H), 1.45-1.81(m, 10H), 1.91(d, J=12.5 Hz, 1H), 2.17(s, 1H), 3.02(s, 1H), 3.21(s, 3H), 3.84(s, 3H), 6.94(d, J=9.0 Hz, 1H), 7.53(br s, 2H), 7.33 ppm (t, J=8.0 Hz, 1H); IR(KBr) 2918, 1611, 1512, 1175 cm⁻¹

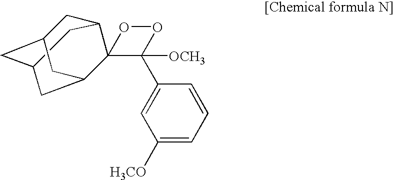

### 3-(2′-Spiroadamantane)-4-methoxy-4-(3″-methoxy)phenyl-1,2-dioxetane

¹H NMR(500 MHz, CDCl₃) 1.03 (d, J=12.0 Hz, 1H), 1.24(d, J=12.0 Hz, 1H), 1.45-1.90(m, 10H), 2.12(s, 1H), 3.04(s, 1H), 3.23(s, 3H), 3.85(s, 3H), 6.94(d, J=8.0 Hz, 1H), 7.18(br s, 2H), 7.33 ppm (t, J=8.0 Hz, 1H); IR(KBr) 2920, 2860, 1586 cm⁻¹

### Test Example 4

[Measurement II]

Measurement of reaction heat: heat generated from above-mentioned compounds was measured by the differential thermal analyzer in accordance with above-mentioned [Measurement I]. Thermal measurement of a dioxetane compound in the solid state was carried out as follows: 2 to 3 mg of the dioxetane compound was weighed, filled into an aluminum capsule, and heated gradually to 80-180° C. by DSC-50 (Shimadzu Corporation) to measure the generated heat. Results obtained are shown in Table 3.

TABLE 3
Reaction heat of dioxetanes and survival rate of cancer cells

| | Solid reaction | Survival rate of |
|---|---|---|
| Entry | heat/kcal/mol | cells % (100 μM)^{a} |
| Chemical formula M | 68.8 | 55 |
| Chemical formula N | 66.8 | 67 |

^{a}Concentration of Chemical formula M and Chemical formula N for MTT assay

### Test Example 5

[Anticancer Effect II]

Results of measurements by MTT assay shown in [Anticancer effect I] are shown in Table 3.

### Example 5

Compounds of [Chemical formula O] and [Chemical formula P] shown below were synthesized in accordance with Example 1, Example 2 and Example 3. Specifically, terephthalaldehyde (0.340 g, 2.54 mmol), benzil of [Chemical formula g] (1.03 g, 4.90 mmol) and ammonium acetate (3.88 g, 50.4 mmol) were reacted in acetic acid (60 mL) to obtain a crude product, which was then recrystallized from 1,4-dioxane or DMAc-H₂O to obtain compound of [Chemical formula l] as colorless powder (1.18 g, 93%).

Results of analysis of compound of [Chemical formula l] m.p. >300° C. (IPE Letters, vol. 3, p. 30-34(2002), 410-412° C.); ¹H NMR(300 MHz, DMSO-d₆) δ 7.22-7.44(m, 12H), 7.45-7.59(m, 8H), 8.18(s, 4H), 12.8(br s, 1H); FT-IR(KBr) υmax 1605(C═N), 1489, 1444, 843, 766, 696 cm−1; UV(DMSO) λmax (log ε) 304 (sh) (4.32), 362 (4.69) nm; MS (m/z, FAB) 515(M+1); HRMS (FAB) Observed m/z 515.2238 ([M+H]⁺), Calcd. for C₃₆H₂₇N₄ 515.2236. Elemental Analysis Calcd. for C₃₆H₂₆N₄: C 84.02; H 5.09; N, 10.89. Found: C, 83.33; H, 5.11; N, 10.80.

The compounds of [Chemical formula l] (77.2 mg, 0.150 mmol) was added with methylene blue, and irradiated with artificial daylight while blowing oxygen for 13 hours to obtain the compound of [Chemical formula O] (43.4 mg, 50%) as pale yellow powder.

Results of analysis of compound of [Chemical formula O] m.p. 108° C. (dec.); ¹H NMR(300 MHz, DMSO-d₆) δ 7.25-7.70(m, J=7.2 Hz, 16H), 8.13(d, J=7.2 Hz, 4H), 8.54(m, 4H), 12.7(br s, 2H); FT-IR(KBr) υmax 1607(C═N), 1560.

The compound of [Chemical formula O] was subjected to t-butyldimethylsilylation to obtain the compound of [Chemical formula P] as colorless powder.

Results of analysis of compound of [Chemical formula P] m.p. 177-182° C. (dec.); ¹H NMR(500 MHz, CDCl₃) δ 0.17(s, 6H), 0.20(s, 6H), 0.84(s, 18H), 7.28-7.32(m, 6H), 7.33-7.37(m, 4H), 7.45(t, J=7.5 Hz, 4H), 7.53(t, J=7.5 Hz, 2H), 8.25(d, J=7.5 Hz, 4H), 8.61(s, 4H).

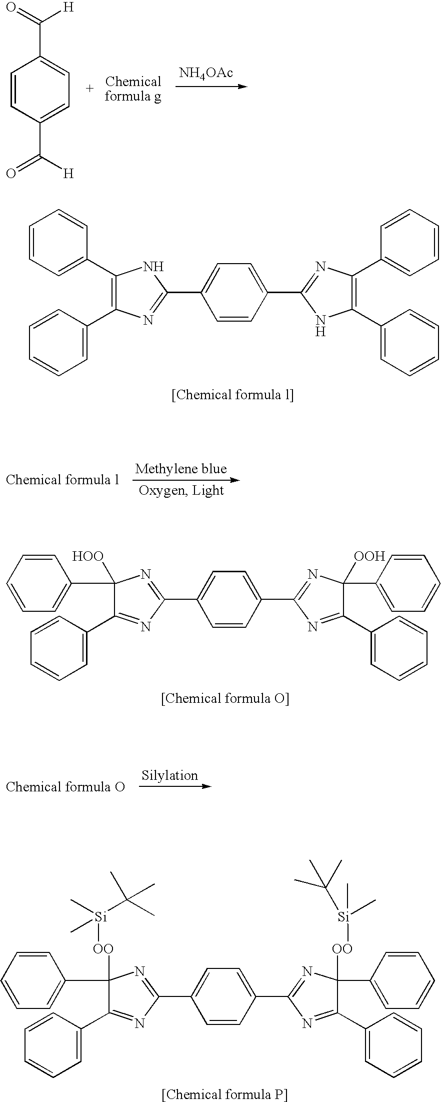

[Chemical formula O] had a generation heat of 47.1 Kcal/mol, and [Chemical formula P] had a melting point of 177-182° C. (decomposition), a generation heat of 147 Kcal/mol, and an imidazole yield of 41%.

### Example 6

The compounds of [Chemical formula Q] and [Chemical formula R] shown below were synthesized in accordance with Example 1, Example 2 and Example 3. Isophthalaldehyde (0.275 g, 2.05 mmol), benzil of [Chemical formula g] (1.02 g, 4.85 mmol) and ammonium acetate (6.65 g, 86.2 mmol) were reacted in acetic acid (40 mL) to obtain a crude product, which was then recrystallized from ethyl acetate to obtain the compound of [Chemical formula m] (0.847 g, 69%) as a colorless needle.

Results of analysis of compound of [Chemical formula m]

m.p. 294-296° C.; ¹H NMR(300 MHz, DMSO-d₆) δ 7.20-7.47(m, 12H), 7.50-7.61(m, 9H), 8.07(d, J=7.7 Hz, 2H), 8.80(s, 1H), 12.8(br s, 2H); FT-IR(KBr) υmax 1603(C═N), 1485, 1456, 762, 694 cm⁻¹; UV(DMSO) λmax (log ε) 315(4.75) nm; MS (m/z, FAB) 515(M+1); HRMS (FAB) Observed m/z 515.2233 ([M+H]⁺), Calcd. for C₃₆H₂₇N₄ 515.2236. Elemental Analysis Calcd. for C₃₆H₂₆N₄.C₄H₈O₂: C, 79.71; H, 5.69; N, 9.30. Found: C, 79.49; H, 5.63; N, 9.37.

The compound of [Chemical formula m] (216 mg, 0.358 mmol) was added with methylene blue, and irradiated with artificial daylight while blowing oxygen for seven hours to obtain the compound of [Chemical formula Q] (176 mg, 90%) as colorless powder.

Results of analysis of compound of [Chemical formula Q]

m.p. 129-132° C. (dec.); ¹H NMR(MHZ, DMSO-d₆) δ 7.21-7.93(m, 17H), 8.14(d, J=7.3 Hz, 4H), 8.56(J=8.7, 2 Hz, 2H), 9.28(d, J=2 Hz, 1H); FT-IR(KBr) υmax 1618.

The compound of [Chemical formula Q] was subjected to t-butyldimethylsilylation to obtain the compound of [Chemical formula R].

Results of analysis of compound of [Chemical formula R]

m.p. 52-61.5° C.; ¹H NMR(200 MHz, CDCl₃) δ 0.15(s, 6H), 0.18(s, 6H), 0.83(s, 18H), 7.20-7.70(m, 17H), 8.25(d, J=8.0 Hz, 4H), 8.60(m, 2H), 9.52(m, 1H).

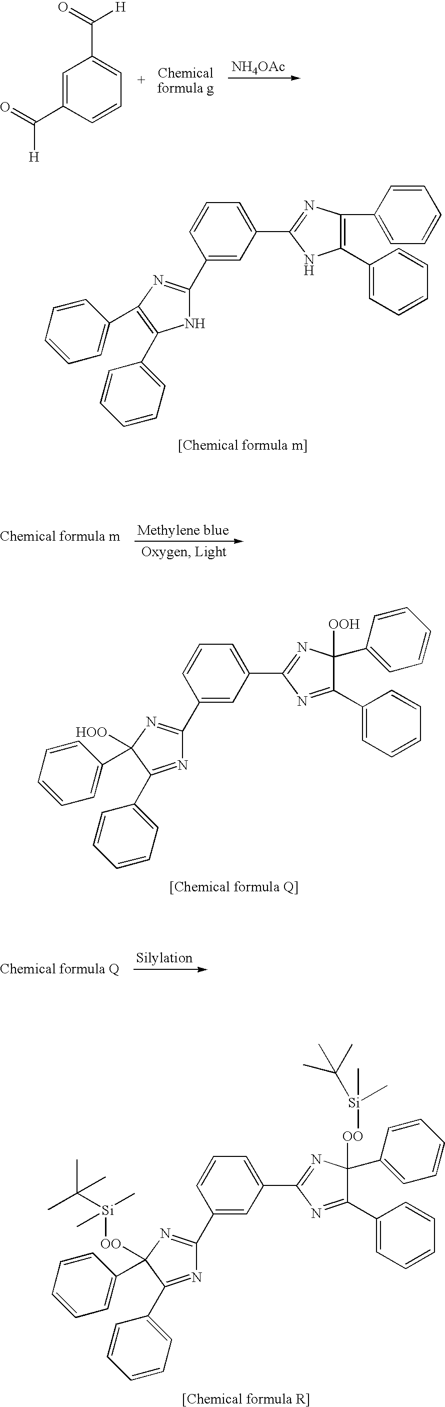

[Chemical formula Q] had a generation heat of 30.5 Kcal/mol and [Chemical formula R] had a generation heat of 143 Kcal/mol.

### INDUSTRIAL APPLICABILITY

The heat generator according to the present invention has a reaction heat of about 20 Kcal/mol to 90 Kcal/mol and/or a singlet oxygen yield of approximately 50%, and the pharmaceutical composition comprising the same hardly develops side effects and is hardly tolerated, imposes little burden on patients, and exhibits high anticancer activity.

1. A method of treating cancer or inducing sudden death of cancer cells by administering to a patient in need thereof a pharmaceutically effective amount of a heat- and/or singlet oxygen-generating agent comprising a compound selected from the group consisting of an organic peroxide and a chemiluminescent compound.

2. (canceled)

3. The method according to claim 1, wherein the generating agent generates heat and/or singlet oxygen under the environment of a site where cancer cells are present.

4. The method according to claim 1, wherein an incorporation of the generating agent into the cancer cells is accelerated.

5. The method according to claim 1, wherein the compound is an organic peroxide which is a peroxide of an imidazole derivative.

6. The method according to claim 1, wherein the compound is a chemiluminescent compound which is a dioxetane compound.

7. A pharmaceutical composition for cancer treatment comprising an organic peroxide or a chemiluminescent compound, which generates heat and/or singlet oxygen, in combination with a suitable carrier.

8. A pharmaceutical composition for inducing sudden death of cancer cells comprising an organic peroxide or a chemiluminescent compound, which generates heat and/or singlet oxygen, in combination with a suitable carrier.

9. A compound selected from the group consisting of

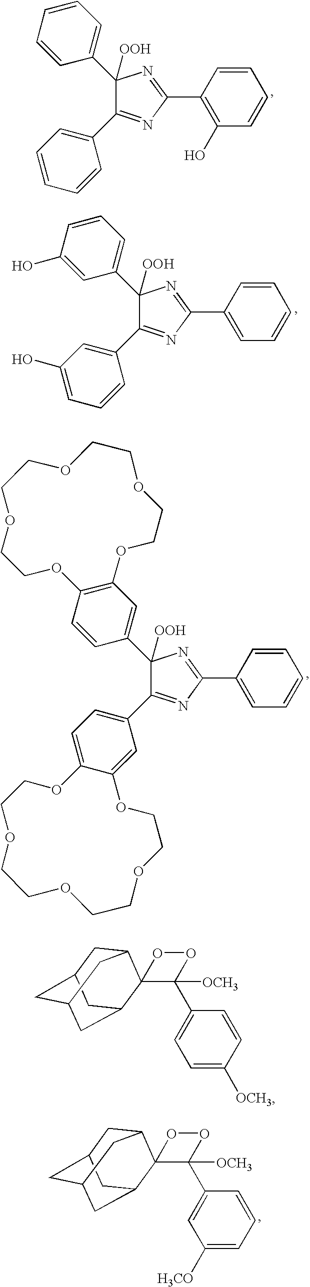

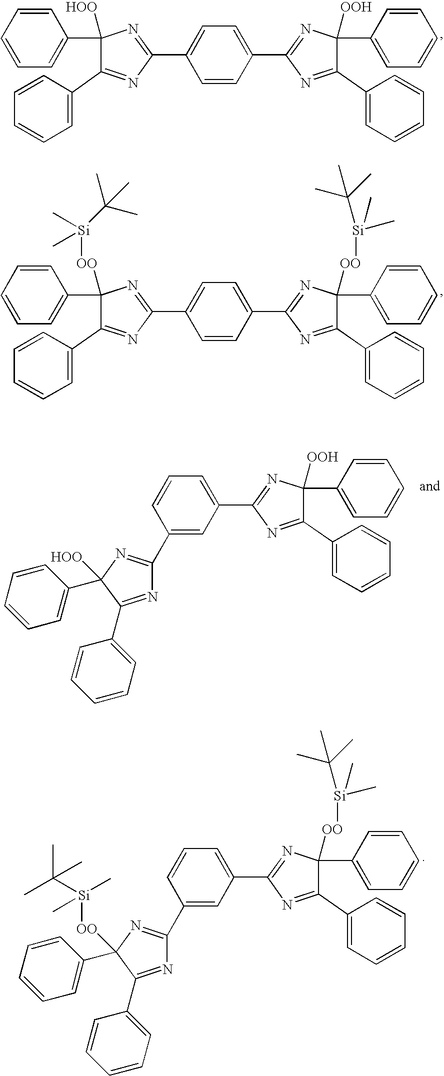

10. The method according to claim 4, wherein the compound is an organic peroxide which is a peroxide of an imidazole derivative.

11. The method according to claim 4, wherein the compound is a chemiluminescent compound which is a dioxetane compound.

12. The method according to claim 3, wherein the compound is an organic peroxide which is a peroxide of an imidazole derivative.

13. The method according to claim 3, wherein the compound is a chemiluminescent compound which is a dioxetane compound.

14. The method according to claim 3, wherein an incorporation of the generating agent into cells is accelerated.

15. The method according to claim 14, wherein the compound is an organic peroxide which is a peroxide of an imidazole derivative.

16. The method according to claim 14, wherein the compound is a chemiluminescent compound which is a dioxetane compound.

17. The method according to claim 1, wherein the method is for treating cancer, said cancer being selected from the group consisting essentially of liver cancer, lung cancer, stomach cancer, large intestine cancer, skin cancer and uterine cancer.

18. The method according to claim 17, wherein the compound is selected from the group consisting of

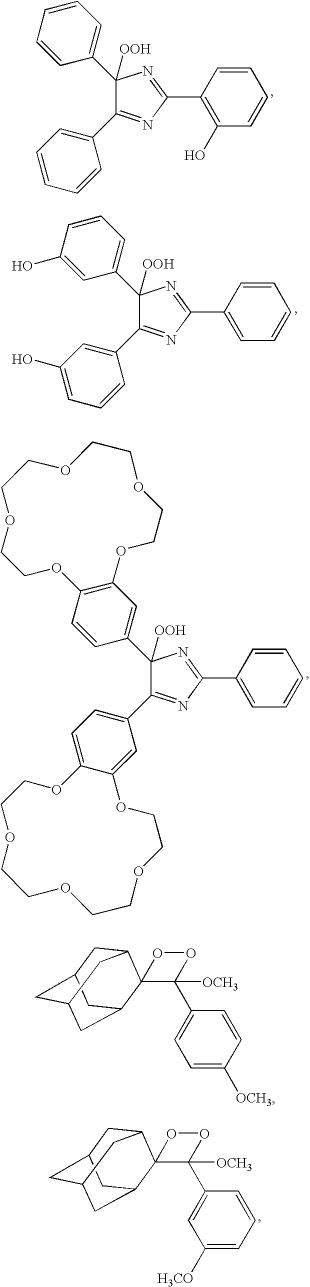

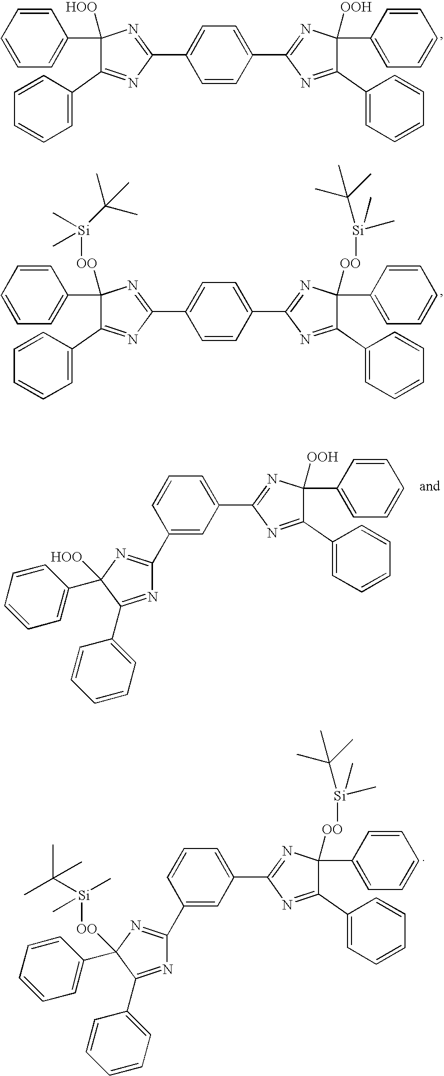

19. The method according to claim 18, wherein said cancer is selected from the group consisting essentially of liver cancer, lung cancer, stomach cancer, large intestine cancer, skin cancer and uterine cancer.
